(19) United States
(12) Patent Application Publication
(10) Pub. No.: US 2003/0212392 A1
(43) Pub. Date: Nov. 13, 2003

(54) Ultrasonic soft tissue cutting and coagulation systems having a curvilinear blade member and clamp

(76) Inventors: Paul Fenton, Marblehead, MA (US); Francis Harrington, Peabody, MA (US); Paul Westhaver, Newburyport, MA (US)

Correspondence Address: Mark G. Lappin, McDermott, Will & Emery, 28 State Street, Boston, MA, 02109, US

(21) Appl. No.: 10/437,223

(22) Filed: May 13, 2003

Related U.S. Application Data
(60) Provisional application No. 60/380,178, filed on May 13, 2002.

(51) Int. Cl. A61B 18/04

(52) U.S. Cl. 606/28

(57) ABSTRACT

Ultrasonic soft tissue cutting or coagulating systems are disclosed that include an ultrasonic blade member for cutting and/or coagulating tissue, and an opposed clamp member which is used together with the blade member to compress/clamp the tissue being treated. At least one of the blade member and the clamp member has a substantially curvilinear configuration. The ultrasonic blade member and the opposing clamp member can have substantially curvilinear and dissimilar configurations which enables soft tissue to be treated evenly across the contact surface

### CROSS-REFERENCE TO RELATED APPLICATIONS

The present application claims priority to provisional U.S. patent application Ser. No. 60/380,178, filed on May 13, 2002, which is assigned to the assignee of the present application and incorporated herein by reference.

### STATEMENT REGARDING FEDERALLY SPONSORED RESEARCH

Not Applicable

### REFERENCE TO MICROFICHE APPENDIX

Not Applicable

### BACKGROUND OF THE INVENTION

For many years, ultrasonic surgical instruments have been used for soft tissue cutting and coagulation. These ultrasonic instruments include ultrasonic transducers which convert the electric energy supplied by a generator into ultrasonic frequency vibratory energy, which can then be applied to the tissue of a patient. Ultrasonic surgical instruments use relatively high-power, low-frequency vibratory energy, typically at a frequency range of about 20 kHz to about 100 kHz.

In general, ultrasonic tissue cutting and coagulation systems include a member that is coupled to the ultrasonic transducers, and that can be made to vibrate at ultrasonic frequencies. The ultrasonically vibrating member, for example a surgical blade, is then applied to the tissue, in order to transmit ultrasonic power to the tissue. In this way, the contacted tissue can be cut or coagulated. Ultrasonic surgical systems offer a number of advantages over conventional surgical systems, for example reduction of bleeding and trauma.

The mechanism through which an ultrasonically vibrating member and the tissue interact, i.e. the physics of ultrasonic soft tissue cutting and coagulation, is not completely understood, however various explanations have been provided by researchers over the years. These explanations include descriptions of mechanical effects and thermal effects. The mechanical viewpoint states that the tip of the ultrasonically vibrating member generates short-range forces and pressures, which are sufficient to dislodge cells in the tissue, and break up the tissue structures. Various types of forces are postulated as contributing to the rupture of the tissue layer, for example the impact forces resulting from the direct contact of the vibrating tip with tissue, and the shear forces that are the result of the differences in force levels across tissue boundaries. Some energy may be lost due to frictional heating, and due to the heating caused by the absorption of acoustic energy by tissue.

Thermal effects may include frictional heat, generated by the ultrasonically vibrating tip, in an amount sufficient to melt a portion of the contacted tissue. Alternatively, the tissue may absorb the vibratory energy, which it then converts into heat. The generated heat may be used to coagulate a blood vessel, by way of example. Other effects that have been postulated in order to explain the probe-tissue interaction include cavitational effects. The cavitation viewpoint postulates that the coupling of ultrasonic power onto tissue results in the occurrence of cavitation in tissue, namely the formation of gas or vapor-filled cavities or bubbles within the tissue, which may oscillate and propagate. A combination of mechanical, thermal, and cavitational effects may result in the desired surgical outcomes, such as cutting and coagulation.

A number of ultrasonic soft tissue cutting and coagulating systems have been disclosed in the prior art. For example, U.S. Pat. No. 5,322,055 (the “'055 patent”), entitled “Clamp Coagulator/Cutting System For Ultrasonic Surgical Instruments,” discloses ultrasonic surgical instruments having a non-vibrating clamp for pressing tissue against an ultrasonically vibrating blade, for cutting, coagulating, and blunt-dissecting of tissue. The '055 patent issued to T. W. Davison et al. on Jun. 21, 1994, and is assigned on its face to Ultracision, Inc.

The ultrasonic surgical instruments disclosed in the '055 patent include a handpiece enclosing an ultrasonic transducer is connected to the blade. When ultrasonically activated, the blade undergoes longitudinal mode vibrations, parallel to the blade edge. A clamp accessory, including a clamp member, is releasably connected to handpiece. The blade is used in conjunction with the clamp member, to apply a compressive force to the tissue in a direction normal to the direction of vibration. In a preferred embodiment of the invention, a clamp member actuation mechanism, for example a scissors-like grip, actuates a pivoted clamp member to compress and bias tissue against the ultrasonic power-carrying blade, in a direction normal to the longitudinal vibratory movement of the blade.

U.S. Pat. No. 6,036,667 (the “'667 patent”), entitled “Ultrasonic Dissection and Coagulation System,” issued to R. Manna et al. on Mar. 14, 2000, and is assigned on its face to United States Surgical Corporation and to Misonix Incorporated. The '667 patent discloses an ultrasonic dissection and coagulation system for surgical use. The ultrasonic system includes a housing, and an elongated body portion extending from the housing. The housing encloses an ultrasonic transducer, which is operatively connected to a cutting blade by a vibration coupler. The cutting blade has a cutting surface which is angled with respect to the longitudinal axis of the elongated body portion, i.e. with respect to the axis of ultrasonic vibration. A clamp member is movable from an open position in which the operative surface of the clamp is spaced from the cutting surface of the blade, to a clamped position in which the operative surface of the clamp is in close juxtaposed alignment with the cutting surface to clamp tissue therebetween.

U.S. Pat. No. 6,056,735 (the “'735 patent”), entitled “Ultrasound Treatment System,” relates to ultrasonic treatment systems, including endoscopic systems and aspiration systems, for treating living tissue. The '735 patent issued to M. Okada et al. on May 2, 2000, and is assigned on its face to Olympus Optical Co., Ltd. In the ultrasonic treatment system featured in the '735 patent, a handpiece encloses ultrasonic transducers, and a probe is connected to the transducers and serves as an ultrasonic power conveying member. A treatment unit of the ultrasonic treatment system includes a stationary, distal member, to which ultrasonic vibrations are conveyed by the probe, and a movable, holding member. The holding member clamps living tissue, in cooperation with the fixed distal member. A scissors-like manipulating means manipulates the treatment unit to clamp or free living tissue. In a preferred embodiment, a turning mechanism is provided for turning the treatment unit relative to the manipulating means, with the axial direction of the transducers as a center.

The shape and design of the ultrasonically blade member, and in pertinent cases the shape and design of the clamp member used to grasp tissue in cooperation with the blade member, significantly affect the interaction of an ultrasonic surgical system with tissue. The prior art ultrasonic systems described above do not disclose ultrasonically blade members and/or clamp members which have curvilinear configurations that ensure a substantially uniform delivery of ultrasonic power to the tissue that is in contact with the operative surface of the blade member.

It is desirable to provide an ultrasonic surgical system which enables soft tissue to be treated evenly across the contact surface, thereby improving the coupling of ultrasonic power to the tissue. It is also desirable to provide an ultrasonic surgical system which enables tissue to be treated according to a desired spatial distribution of ultrasonic power across the contact surface.

### SUMMARY OF THE INVENTION

The present invention relates to ultrasonic soft tissue cutting or coagulating systems that include an ultrasonic blade member for cutting and/or coagulating tissue, and an opposed clamp member which can be used together with the blade member to compress/clamp the tissue being treated. At least one of the blade member and the clamp member has a substantially curvilinear configuration. This curvilinear configuration can be optimized to improve the coupling of ultrasonic power to the tissue being treated.

An ultrasonic surgical instrument constructed in accordance with one embodiment of the present invention includes one or more ultrasonic transducers for generating ultrasonic vibrations. An elongated ultrasonic transmission coupler includes a proximal end and a distal end, and is connected to the ultrasonic transducer at the proximal end. The transmission coupler receives ultrasonic vibrations from the transducer, and transmits these ultrasonic vibrations from its proximal end to its distal end.

An ultrasonic surgical assembly is connected to the distal end of the elongated transmission coupler. In one embodiment, the assembly includes a blade member, and a clamp member. The blade member and the clamp member are movably connected, and cooperate to engage tissue between their respective operative surfaces. In one embodiment, the blade member is acoustically coupled to the transmission coupler so as to receive ultrasonic power from the coupler. Upon receipt of ultrasonic power, the blade member undergoes vibratory motion. The blade member of the ultrasonic surgical assembly thereby delivers ultrasonic power to contacting tissue, so that desired surgical effects, such as cutting and/or coagulation, can be achieved.

In another embodiment of the invention, the clamp member may also be acoustically coupled to the transmission coupler, and undergo vibratory motion upon receipt of ultrasonic power. In this embodiment, either the blade member or the clamp member, or both, may vibrate ultrasonically.

In the present invention, at least one of the blade member and the clamp member are characterized by a substantially curvilinear configuration. In one embodiment of the invention, the curvilinear configuration of the blade member and/or the clamp member enables ultrasonic power to be substantially uniformly delivered to the tissue, across the length of the contact surface. In another embodiment of the invention, the curvilinear configuration of the blade member and/or the clamp member permits the delivery of ultrasonic power according to a desired spatial distribution.

In one form of the invention, the blade member is rigidly attached to the transmission coupler, and the clamp member is movably attached to the coupler. In this embodiment, the clamp member is movable from an open position in which the blade member and the clamp member are spaced apart, to a closed position in which the blade member and the clamp member are in engagement so as to grasp tissue therebetween. In an alternative form of the invention, the clamp member is rigidly attached to the transmission coupler, and the blade member is movably attached to the coupler, and is movable from the open position to the closed position.

In another form of the invention, a scissors-like blade-clamp assembly for an ultrasonic surgical system has a moveable blade member and a moveable clamp member, in which opposing lateral surfaces of the moveable blade member and the moveable clamp member are adapted for angled interference in response to relative motion therebetween.

### BRIEF DESCRIPTION OF THE DRAWINGS

The invention can be more fully understood by referring to the following detailed description taken in conjunction with the accompanying drawings, in which:

FIG. 1 illustrates an overall schematic view of an ultrasonic surgical system, constructed in accordance with the present invention.

FIG. 2 is a schematic illustration of the velocity distribution and the coupling force distribution resulting from curvilinear and dissimilar configurations of an ultrasonically blade member and a clamp member, in which the geometrical configurations are optimized so as to permit a substantially uniform delivery of ultrasonic power to the tissue.

FIGS. 3A-3D illustrate one embodiment of an ultrasonic surgical assembly, in which a ultrasonic blade member and a receiving clamp member have operative surfaces characterized by curvilinear configurations.

FIGS. 4A-4C illustrate an ultrasonic surgical assembly in which a stationary blade member has an operative surface that is substantially convex-shaped.

FIGS. 5A-5C illustrate an ultrasonic surgical assembly in which a movable blade member has an operative surface that is substantially convex-shaped.

FIGS. 6A-6D illustrate a scissors-like blade-clamp assembly for an ultrasonic surgical system, in which opposing lateral surfaces of a moveable blade member and a moveable clamp member are adapted for angled interference in response to relative motion therebetween.

FIG. 7 illustrates an ultrasonic surgical assembly in which the blade member and the clamp member have a serrated configuration.

FIG. 8 schematically illustrates the sinusoidal functions that represent the geometrical variations of the operative surfaces of a blade member and a clamp member that have serrated configurations.

FIG. 9 schematically illustrates an ultrasonic surgical assembly in accordance with one embodiment of the present invention, in which the blade member is movable toward the clamp member in a direction parallel to the longitudinal vibrations of the blade member, and no scissors-type mechanism is needed.

### DETAILED DESCRIPTION

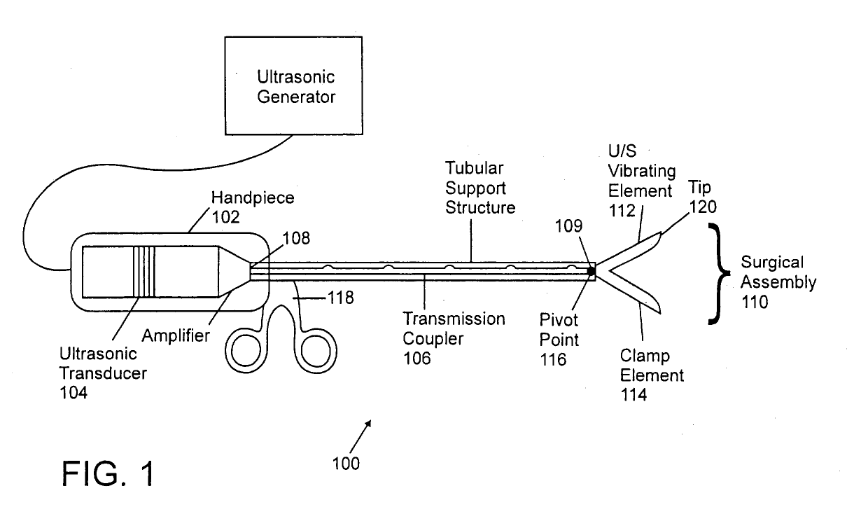

FIG. 1 illustrates an overall schematic view of an ultrasonic soft tissue cutting and coagulating system 100, constructed in accordance with one embodiment of the present invention. The system include a handpiece 102 that encloses one or more ultrasonic transducers 104. An ultrasonic generator is connected to the handpiece 102, and supplies electric energy. The transducers 104 convert the supplied electric energy into ultrasonic frequency vibratory energy. The frequency range at which the system 100 operates is typically between about 20 kHz and about 100 kHz, and the electric power supplied by the ultrasonic generator is typically between about 100 W to about 150 W, although other frequencies and power levels can be used. The ultrasonic transducers 104 may be made of piezoelectric material, or may be made of other materials, such as nickel, that are capable of converting electric energy into vibratory energy. The handpiece 102 may also enclose an amplifier, for example an acoustic horn, which amplifies the mechanical vibrations generated by the ultrasonic transducers 104.

An elongated ultrasonic transmission coupler 106 is connected to the handpiece 102. In one embodiment, the transmission coupler 106 has a proximal end 108 and a distal end 109, and is connected to the handpiece 102 at the proximal end. The ultrasonic transmission coupler 106 transmits the ultrasonic vibratory energy, received from the transducers 104, from its proximal 108 end to its distal end 109.

In the illustrated embodiment, an ultrasonic surgical assembly 110 is connected to the distal end 109 of the elongated transmission coupler 106, and includes an ultrasonic blade member 112, and a clamp member 114. In a preferred embodiment, the blade member 112 and the clamp member 114 are movably connected to each other, and cooperate to engage tissue between their respective operative surfaces. In the illustrated embodiment, the blade member 112 is acoustically coupled to the transmission coupler 106, so that the ultrasonic power is transmitted to, and carried by, the blade member 112. The blade member 112 undergoes vibratory motion upon receipt of ultrasonic vibrations from the transducer(s) 104, and thereby delivers ultrasonic power to contacting tissue, so that desired surgical effects, such as cutting and/or coagulation, can be achieved.

In another embodiment of the invention (not shown), the clamp member 114 may also be acoustically coupled to the transmission coupler 106, so that the ultrasonic power can also be transmitted to, and carried by, the clamp member 114. In this embodiment, either the blade member 112 or the clamp member 114, or both, may vibrate ultrasonically.

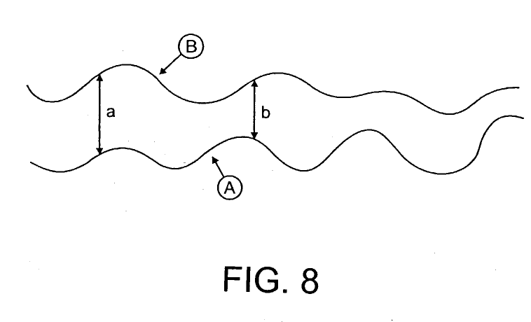

The blade member 112 and the clamp member 114 may be pivotally mounted at the end of the elongated transmission coupler 106, about a pivot point 116, although in other embodiments of the invention (for example the embodiment illustrated in FIG. 8 below), other mechanisms for movably connecting the blade member 112 and the clamp member 114 may be used. In the illustrated embodiment illustrated in FIG. 1, the surgical assembly 110 is activated by a scissors-like clamp activation mechanism 118.

The ultrasonic system 100 is generally characterized by a resonant frequency, which is determined primarily by the assembled length of its components. The most efficient vibrations occur when the ultrasonic system 100, including the handpiece 102, the transmission coupler 106, and the surgical assembly 100, is vibrated at its intended resonant frequency. In this case, the maximum vibratory motion occurs at the tip 120 of the blade member 112.

The shape and design of the blade member 112, as well as the clamp member 114, significantly affect the interaction of the ultrasonic surgical system 100 with tissue. In the present invention, at least one of the ultrasonic blade member 112 and the receiving clamp member 114 has a substantially curvilinear configuration. The blade member 112 and the clamp member 114 are movable relative to each other, between an open position in which the blade member 112 and the clamp member 114 are spaced apart, and a closed position in which the blade member 112 and the clamp member 114 are in engagement so as to capture tissue between their respective operative surfaces.

In a preferred embodiment of the invention, the operative surfaces of the blade member 112 and the receiving clamp member 114 are not only curvilinear, but also dissimilar. In other words, at least portions of the respective operative surfaces of the blade member and the clamp are characterized by substantially different curvature rates. The spacing between the respective surfaces is non-uniform, and varies over portions of, or over all points between, one end of the surgical assembly 110 to the other. In this description, and henceforth in this specification, the word “dissimilar” is used in the sense of the antonym of “similar,” as used when saying that two polygons are not “similar,” where a “similar” polygon is generally defined as two polygons whose corresponding angles are congruent, and whose corresponding sides are proportional, as can be found in geometry textbooks.

The curvilinear and dissimilar configurations for the blade member 112 and the clamp member 114 result in several advantageous features for the ultrasonic surgical system 100, as compared to prior art ultrasonic systems that have linear and/or parallel blade member 112 and clamp member 114. For example, a curvilinear configuration for the blade member 112 and/or clamp member 114 can be optimized so as to produce a substantially uniform distribution of the ultrasonic vibratory energy across the operative surface of the blade member 112. In this way, a substantially uniform cutting/coagulation energy can be delivered along the length of the contact surface with the tissue. The curvilinear configuration can also be optimized so as to achieve a desired spatial distribution of ultrasonic power along the length of the contact surface. Finally, a curvilinear clamp member 114 that is offset and dissimilar to the blade member has, in some forms of the invention, a greater tissue-grasping potential as compared to linear or parallel clamp members known in the prior art.

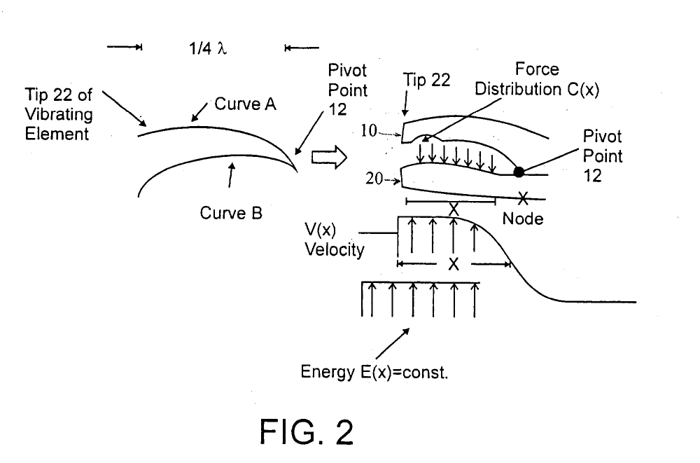

FIG. 2 is a schematic illustration of the velocity distribution, the coupling force distribution, and the ultrasonic power distribution, which result from an ultrasonic blade member and a clamp member that have curvilinear and dissimilar configurations that are optimized so as to permit a substantially uniform delivery of ultrasonic power to the tissue. In the illustrated embodiment, the blade member 10 and the clamp member 20 are pivotally mounted about a pivot point 12. In this embodiment, the ultrasonic vibrations of the blade member 10 are characterized by a resonant frequency at which the maximum vibratory motion occurs at a tip 22 of the blade member 10, and at which a vibratory node occurs at the pivot point 12. The distance between the tip 22 and the pivot point 12 is thus given by (¼)(λ), where λ represents the wavelength of the ultrasonic vibrations.

Curves A and B in FIG. 2 schematically represent the curvilinear geometrical configurations of the operative surfaces of the blade member 10 and the clamp member 20, respectively. Curve V(x) in FIG. 2 schematically represents the spatial variation of the transverse velocity of the blade member 20, along its operative surface. Curve C(x) in FIG. 2 schematically represents the ultrasonic coupling to the tissue being treated, i.e. the mechanical compressive force exerted on the tissue by the operative surfaces of the ultrasonically blade member 10 and the clamp member 20. As seen in FIG. 2, the coupling force C(x) is maximum at the pivot point 12 (i.e. the vibratory node), while the velocity V(x) of the blade member 10 is a minimum at the pivot point 12 and a maximum at the tip 22.

In the present invention, it is recognized that the geometrical variations A and B of the operative surfaces of the blade member 10 and the clamp member 20 can be controlled in such a way that the distribution V(x) of the transverse velocity of blade member 10 along the length of its operative surface can be accounted for. In particular, in the illustrated embodiment the geometrical variations A and B of the operative surfaces of the blade member 10 and the clamp member 20 are made in such a way that the product of 1) the transverse velocity V(x) of the blade member 10 and 2) the mechanical coupling force C(x) is constant, at every point x along the contact surface between the blade member 10 and the tissue. In this way, a substantially uniform distribution of ultrasonic power can be achieved along the entire length of the blade member 10, as shown by curve E(x)=constant, which schematically represents the resulting spatial distribution of ultrasonic power that is delivered to the contacted tissue.

In an alternative embodiment (not shown), the geometrical variations of the operative surfaces of the blade member and the clamp member can be controlled in such a way that the product of the transverse velocity V(x) of the blade member and the coupling force C(x) has a desired and predetermined spatial dependence along the contact surface between the blade member and the tissue, i.e.:

(velocity V(x) of blade member)*(coupling force C(x) )=f_{E}(x),

where f_{E}(x) represents the spatial distribution of the ultrasonic power delivered to the tissue.

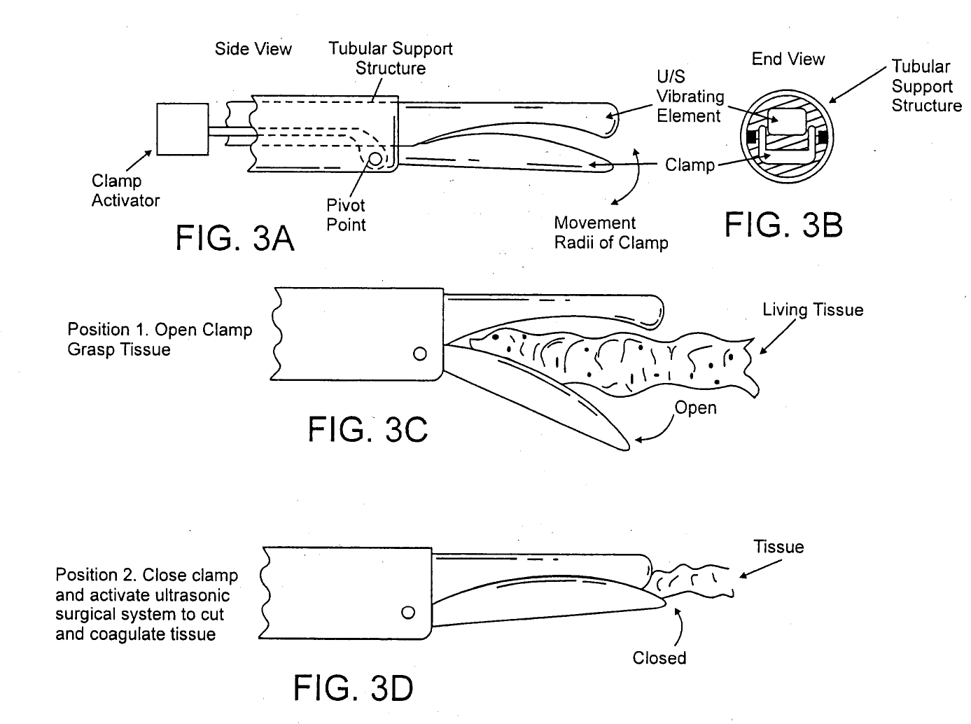

FIGS. 3A-3D illustrate one embodiment of a surgical assembly for an ultrasonic system in which both a ultrasonic blade member and a receiving clamp member have operative surfaces characterized by curvilinear configurations. FIG. 3A provides a side view of the surgical assembly, while FIG. 3B provides an end view thereof. In the illustrated embodiment, the clamp member is pivotally mounted at the end of a tubular support structure, about a pivot point. The pivot point is shown as being disposed at a location remote from the tip of the ultrasonic blade member. A clamp activator, shown schematically in block diagram form in FIG. 3A, may be provided in order to activate the pivotally connected blade member and the receiving clamp member.

FIG. 3C illustrates an open-clamp configuration, while FIG. 3D illustrates a closed-clamp configuration, for the surgical assembly illustrated in FIGS. 3A-3D. As seen from FIGS. 3C and 3D, the blade member and the clamp member are movably connected. In particular, in the illustrated embodiment the blade member is stationary, while the clamp member is movable from an open position (shown in FIG. 3C) in which the clamp member is spaced apart from the blade member, to a closed position (shown in FIG. 3D) in which the contacting tissue is grasped between the operative surfaces of the blade member and the clamp member.

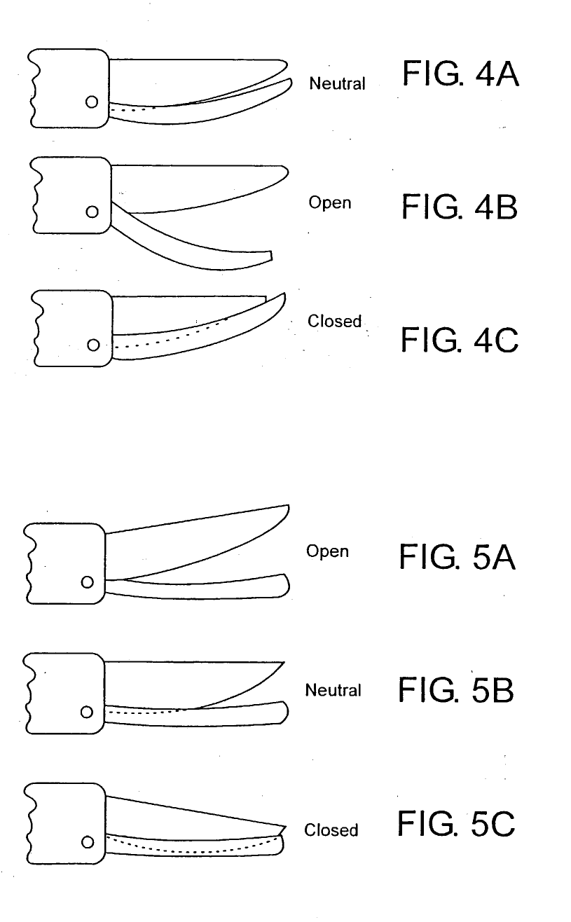

FIGS. 4A-4C illustrate a surgical assembly which includes an ultrasonically blade member has a curvilinear operative surface that is substantially convex-shaped, and the clamp member has a curvilinear operative surface that is substantially concave-shaped. As in FIGS. 3A-3C, the ultrasonic blade member is stationary, while the clamp member is movable. FIG. 4A illustrates a neutral position of the surgical assembly, i.e. a position in which the clamp member is neither maximally spaced apart, nor closed and in engagement against the blade member. FIG. 4B illustrates an open position of the movable clamp member, in which the clamp member is positioned at a location spaced apart from the blade member. FIG. 4C illustrates a closed position of the clamp member, in which tissue can be grasped between the respective operative surfaces of the blade member and the clamp member.

In some surgical procedures, it may be desirable for certain sections of the tissue to receive higher energies, as compared to other sections of the tissue. The ultrasonic vibrational mode along the operative surface of the convex-shaped blade member, illustrated in FIG. 4A-4C, is less uniform, as compared to ultrasonic modes along the operative surface of a linearly shaped blade member. In the convex-shaped blade member, therefore, the ultrasonic vibrational mode can be such that one or more sections of the operative surfaces of the blade member have a higher energy region, for maximum surgical effect. As discussed in conjunction with FIG. 2, this may be accomplished by controlling the geometric variations of the operative surfaces of the blade member and the clamp member in such a way that

V(x)*C(x)=f_{E}(x),

where V(x) is the transverse velocity distribution of the blade member along the operative surface of the blade member, C(x) is the ultrasonic coupling force distribution, and f_{E}(x) is the desired spatial distribution of ultrasonic power along the length of the contact surface between the tissue and the operative surface of the blade member.

FIGS. 5A-5C illustrate a surgical assembly in which the ultrasonic blade member has an operative surface that is substantially curvilinear, and is dissimilar to the operative surface of a curvilinear clamp member. As in the embodiment illustrated in FIGS. 3A-3C, the ultrasonic blade member has an operative surface that is substantially convex-shaped, and the clamp member has an operative surface that is substantially concave-shaped.

In the illustrated embodiment, however, the clamp member is not movable, but stationary, in contrast to the embodiments illustrated in FIGS. 3A-3C, and FIGS. 4A-4C. The ultrasonic blade member is movable between an open position (FIG. 4A), a neutral position (FIG. 4B), and a closed position (FIG. 4C) in which the blade member and the clamp member cooperate to engage tissue between their respective operative surfaces.

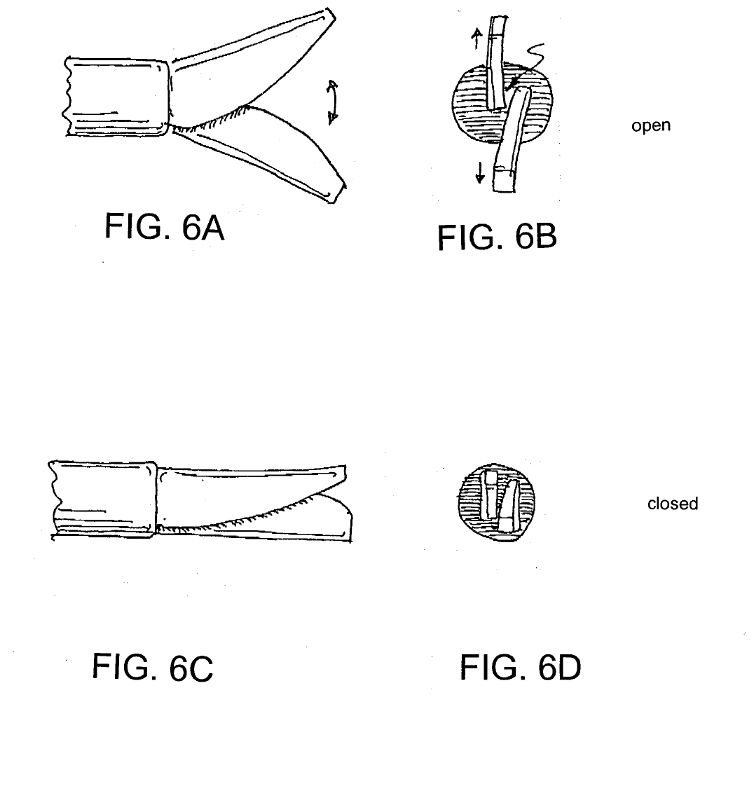

FIGS. 6A-6D illustrate a scissors-like blade-clamp assembly for an ultrasonic surgical system, in which opposing lateral surfaces of a moveable blade member and a moveable clamp member are adapted for angled interference in response to relative motion therebetween. In the illustrated embodiment, the ultrasonically blade member and the clamp member both have curvilinear operative surfaces.

FIG. 6A illustrates an open position of the movable blade member and the moveable clamp member, in which the clamp member is positioned at a location spaced apart from the blade member, while FIG. 6B provides an end view thereof.

FIG. 6C illustrates a closed position of the blade member and the clamp member, in which tissue can be grasped between the respective operative surfaces of the blade member and the clamp member, while FIG. 6D provides an end view thereof.

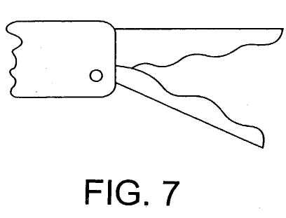

FIG. 7 illustrates a surgical assembly in which the respective operative surfaces of the ultrasonic blade member and the clamp member have a serrated, wave-like configuration. In this embodiment, the operative surface of the blade member may be characterized a substantially sinusoidal configuration, represented by a first sinusoidal wave function f1(x). Likewise, the tissue engaging surface of the clamp member may be characterized by a substantially sinusoidal configuration, represented by a second sinusoidal wave function f2(x). The first sinusoidal wave function and the second sinusoidal wave function may be selected so as to enable a substantially uniform delivery of ultrasonic power to the tissue, or a delivery of ultrasonic power according to a desired spatial distribution.

FIG. 8 schematically illustrates the sinusoidal functions that represent the geometrical variations of the respective operative surfaces of a blade member and a clamp member having serrated configurations, as discussed in conjunction with FIG. 6. In an exemplary embodiment illustrated in FIG. 8, curve A represents f1(x), i.e. the sinusoidally varying geometric configuration of the operative surface of the blade member. Curve B represents f2(x), i.e. the sinusoidally varying geometric configuration of the operative surface of the clamp member. In this embodiment, f1(x) and f2(x) may be given, by way of example, by:

f1(x)=sin(aωx)+sin(ωx), and

f2(x)=[sin(aωx)+sin(ωx)]*sin(bωx),

where ω represents the angular frequency of the sinusoidal variations f1(x) and f2(x), and a and b are parameters that represent the transverse distance between the respective operative surfaces of the blade member and the clamp member, at selected points along the distance x that is measured from one end of the surgical assembly to another. By varying the parameters a and b, the geometrical configurations of the serrated operative surfaces of the blade member and the clamp member can be optimized, in order to achieve a desired energy distribution profile.

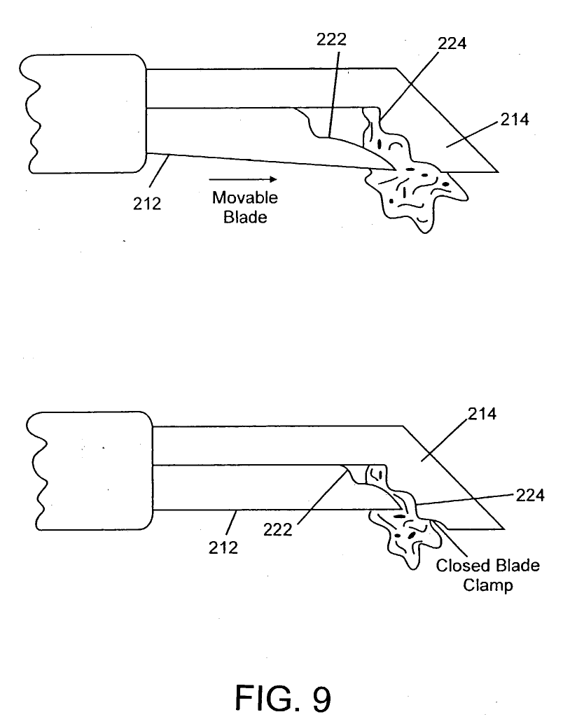

FIG. 9 schematically illustrates one embodiment of the present invention, in which the blade member 212 and the clamp member 214 are movably connected without being pivotally mounted about a pivot point, and without the need of being activated by a scissors-like clamp activation mechanism. In the illustrated embodiment, the blade member 212 and the clamp member 214 are movable relative to each other in a direction parallel to the longitudinal ultrasonic vibrations of the blade member 212. In particular, the movable blade member 212 is connected to the fixed clamp member 214 so that when the blade member 212 is moved in the direction of the longitudinal vibrations, the blade member 212 aligns against the fixed clamp member 214. In this way, tissue disposed between the movable blade member 212 and the fixed clamp member 214 is compressed as the blade member 212 is moved toward the clamp member 214, and the respective opposing surfaces 222 and 224 of the blade member 212 and the clamp member 214 can be used to grasp tissue therebetween. As in the previously discussed embodiments, the operative surfaces 222 and 224 of the blade member 212 and the clamp member 214 are substantially curvilinear. The operative surfaces 222 and 224 of the blade member 212 and the clamp member 214 are also dissimilar, i.e. at least portions of the respective operative surfaces are characterized by substantially different curvature rates.

Although in the illustrated embodiment, the blade member 212 is movable and the clamp member 214 is fixed, in an alternative embodiment (not shown) the blade member 212 may be fixed, and the clamp member 214 may be movable along the direction of the longitudinal ultrasonic vibrations.

In sum, by providing an ultrasonic blade member and an opposing clamp member that have substantially curvilinear and dissimilar configurations, the present invention enables soft tissue to be treated evenly across the contact surface, or in accordance with a desired energy distribution profile. The coupling of ultrasonic power to tissue is thereby improved.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. An ultrasonic surgical instrument, comprising:
a. an ultrasonic transducer for generating ultrasonic vibrations;
b. an elongated ultrasonic transmission coupler having a proximal end and a distal end and connected to said transducer at said proximal end, said coupler being adapted to receive ultrasonic vibrations at said proximal end and transmit said ultrasonic vibrations to said distal end;
c. an ultrasonic surgical assembly connected to said distal end of said coupler, said ultrasonic surgical assembly including a blade member and a clamp member movable relative to each other from an open position in which the blade member and the clamp member are spaced apart, to a closed position in which the blade member and the clamp member are in engagement so as to capture tissue therebetween;
wherein at least one of said blade member and said clamp member are characterized by a substantially curvilinear configuration.

2. An ultrasonic surgical instrument according to claim 1, wherein said blade member is acoustically coupled to said ultrasonic coupler for receiving ultrasonic vibrations therefrom so as to undergo vibratory motion, thereby permitting ultrasonic power to be delivered to tissue in contact with said blade member.

3. An ultrasonic surgical instrument according to claim 1, wherein said blade member is rigidly attached to said coupler, and said clamp member is movably attached to said coupler and is movable toward said rigidly attached blade member from said open position to said closed position.

4. An ultrasonic surgical instrument according to claim 1, wherein said clamp member is rigidly attached to said coupler, and said blade member is movably attached to said coupler and is movable toward said rigidly attached clamp member from said open position to said closed position.

5. An ultrasonic surgical instrument according to claim 1, wherein said blade member has an operative surface characterized by a first curvature rate, and wherein said clamp member has an operative surface characterized by a second curvature rate.

6. An ultrasonic surgical instrument according to claim 5, wherein said first curvature rate and said second curvature rate are substantially different.

7. An ultrasonic surgical instrument according to claim 1,
wherein said blade member includes an operative surface characterized a substantially sinusoidal configuration represented by a first sinusoidal wave function; and
wherein said operative surface of said clamp member is characterized by a substantially sinusoidal configuration represented by a second sinusoidal wave function.

8. An ultrasonic surgical instrument according to claim 1, wherein opposing lateral surfaces of said blade member and said clamp member are adapted for angled interference in response to relative motion therebetween.
